(19) United States
(12) Patent Application Publication
(10) Pub. No.: US 2026/0108514 A1
(43) Pub. Date: Apr. 23, 2026

(54) METHOD OF INHIBITING EPITHELIAL-MESENCHYMAL TRANSITION AND CANCER METASTASIS

(71) Applicant: Agency for Science, Technology and Research, Singapore (SG)

(72) Inventors: Wai Leong TAM, Singapore (SG); Elina PATHAK, Singapore (SG)

(73) Assignee: Agency for Science, Technology and Research, Singapore (SG)

(21) Appl. No.: 18/996,869

(22) PCT Filed: Jul. 21, 2023
(86) PCT No.: PCT/SG2023/050510

§ 371 Date: Jan. 17, 2025

(30) Foreign Application Priority Data
Jul. 21, 2022 (SG) 10202250529T

(51) Int. Cl. A61K 31/505; A61K 31/42; A61K 45/06; A61P 35/04

(52) U.S. Cl. CPC A61K 31/505; A61K 31/42; A61K 45/06; A61P 35/04

(57) ABSTRACT

The invention relates generally to field of cancer biology. Provided herein are methods of inhibiting epithelial-mesenchymal transition (EMT) and for inhibiting or preventing metastasis in a cancer cell, the methods comprising contacting the cancer cell with an effective amount of a histone deacetylase 6 (HDAC6) inhibitor and a glycogen synthase kinase-3[3 (GSK30) inhibitor. Methods of screening for an inhibitor of EMT comprising a reporter cell line, which comprises a ZEB1 inducible construct, and an epithelial gene reporter construct are also provided herein.

### FIELD OF INVENTION

The invention relates generally to the field of cancer biology. Provided herein are methods of inhibiting epithelial-mesenchymal transition (EMT) and for inhibiting or preventing metastasis in a cancer cell.

### BACKGROUND

Cell state transition involves the activation of epithelial-mesenchymal transition (EMT), which is a key determinant of the invasion-metastasis cascade in some cancers. The acquisition of distinct cellular states support the adaptation of cancer cells to various steps of the invasion-metastasis cascade, leading to colonization and the formation of macro-metastases. Despite metastasis being a major cause of cancer therapy failure, its treatment or prevention remains poorly understood.

Cancer, including metastatic cancer, is typically treated with either chemotherapy and/or radiation therapy. Whilst often effective to destroy a significant amount of tumor cells, such therapies often leave behind a number of tumor cells that are resistant to the treatment. These resistant cells can proliferate to form new tumors that are then resistant to treatment. As a result, the constant use of known combinations of chemotherapeutic drugs has given rise to multidrug resistant (‘MDR’) tumor cells.

The mode of proliferative diseases, such as tumors, is multi-factorial. For instance, research over the last forty years has led to the realization that cytotoxic agents (or anti-proliferative agents) includes anti-metabolic agents which interfere with microtubule formulation, alkylating agents which are able to cross-link DNA, platinum based agents which are able to interfere with DNA alkylation by blocking DNA replication, antitumor antibiotic agents, topoisomerase inhibitors, etc. In the treatment of such diseases drugs with different mechanisms may be combined (i.e., combination therapies) with beneficial effects including the effective treatment of MDR tumor cells and to minimize side effects such as undesirable cytotoxicity. The difficulty here is that not all known anti-proliferative agents provide useful or beneficial effects in combination and accordingly research in many laboratories is presently focused on developing new and useful anti-proliferative combination partners.

Accordingly, it is generally desirable to overcome or ameliorate one or more of the above-mentioned difficulties.

### SUMMARY

Disclosed herein is a method of inhibiting epithelial-mesenchymal transition (EMT) in a cancer cell, the method comprising contacting the cancer cell with an effective amount of a histone deacetylase 6 (HDAC6) inhibitor and a glycogen synthase kinase 3β (GSK3β) inhibitor.

Disclosed herein is a method of inhibiting or preventing metastasis of a cancer cell, the method comprising contacting the cancer cell with an effective amount of a histone deacetylase 6 (HDAC6) inhibitor and a glycogen synthase kinase 3β (GSK3β) inhibitor.

Disclosed herein is a method of treating cancer in a subject, the method comprising administering an effective amount of a histone deacetylase 6 (HDAC6) inhibitor and a glycogen synthase kinase 3β (GSK3β) inhibitor to the subject.

Disclosed herein is a method of preventing metastasis of a cancer in a subject, the method comprising administering an effective amount of a histone deacetylase 6 (HDAC6) inhibitor and a glycogen synthase kinase 3β (GSK3β) inhibitor to the subject.

Disclosed herein is a pharmaceutical combination comprising a histone deacetylase 6 (HDAC6) inhibitor, and a glycogen synthase kinase 3β (GSK3β) inhibitor.

Disclosed herein is a method of screening for an inhibitor of epithelial-mesenchymal transition (EMT), the method comprising a) contacting a reporter cell line with a library of candidate inhibitors, so as to identify an inhibitor of EMT in the reporter cell line, wherein the reporter cell line comprises a ZEB1 inducible construct and an epithelial gene reporter construct, wherein the reporter cell line is induced to express ZEB1 to promote EMT, and wherein the epithelial gene reporter construct is capable of reporting the inhibition of EMT in the presence of an inhibitor of EMT.

Disclosed herein is a reporter cell line comprising a ZEB1 inducible construct and an epithelial gene reporter construct.

### BRIEF DESCRIPTION OF DRAWINGS

Embodiments of the present invention are hereafter described, by way of non-limiting example only, with reference to the accompanying drawings in which:

FIG. 1: Design of a cell-based reporter assay. Doxycycline (Dox)-induced ZEB1 driven EMT (HMLE-ZEB1 cells) was used to conduct high-throughput screen for identifying inhibitors of epithelial-mesenchymal transition.

FIG. 2: Identification of inhibitors of epithelial-mesenchymal transition from high-throughput screening of LOPAC library with the screening platform.

A) Dual-Glo luciferase assay to test gene promoters that include CD44, CD166, CDH1 and SERPINE1 in Dox-induced HMLE-ZEB1 cells. Data are represented as ratio of mean Firefly luciferase (FF) and Renilla luciferase (Ren) readings; n=3. B) Dual-glo Luciferase assay of HMLE-ZEB1 screened with LOPAC library. Data are represented as ratio of mean Firefly luciferase (FF) and Renilla luciferase (Ren) readings, n=3. Hits are circled in red. C) Average Z-score of Dual-glo Luciferase assay in HMLE-ZEB1 cells screened with LOPAC library; n=4. D) Validation of screening hits by Dual-glo Luciferase assay in HMLE-ZEB1 screened with LOPAC library by primary and secondary screens. E) Hits from secondary screen validated at different concentrations 5, 1.66, 0.55, 0.18 and 0.06 μM. N=3 F) Validation of the screening hits by CDH1 gene expression. n=3. G) Top, Phase-contrast images of morphological changes of HMLE-ZEB1 cells in DMSO and Dox-treated cells in presence or absence of Kenpaullone (0.25 μM) (DMSO, Dox, Ken, and Dox+Ken). Scale represents 100 μm. Second, Immunofluorescence staining of epithelial marker E-Cadherin (ECAD) and mesenchymal marker Vimentin in DMSO, Dox, Ken and Dox+Ken treated cells. Scale represents 50 μm. Bottom, Fluorescence activated sorting analysis (FACS) of CD44 and CD24 staining in Dox induced HMLE-ZEB1 cells in presence or absence of Ken. Numbers within graph represent percentages of gated cells indicating CD44high CD24low (above) and CD44lowCD24high cells (below). H) TdTomato-labelled MCF7-SLUG+SOX9 cells that were injected orthotopically into the mammary fat pads and induced by Doxycycline (+Dox) to express SLUG and SOX9 to promote metastasis in shControl and shGSK3β cells. Top, Representative fluorescence images of whole-mount of lung and second, histology of lung sections. Scale bar represents 100 μm. Third, Tumors were stained for GSK3β and counterstained with hematoxylin. Scale bar represents 100 μm. Fourth, primary tumors were also stained for E-cadherin, Fibronectin and DAPI. Scale bar represents 50 μm. n=3-5 mice for each group. I) Total number of metastasis as counted from whole mount of lung in mice implanted with in shControl and shGSK3β cells. n=3-5 for each group. **P<0.01 and ***P<0.001 as determined by as determined by one-way ANOVA with Dunnet's multiple comparison test. All data are presented as mean+/−SEM. *P<0.05, **P<0.01, ***P<0.001 and ****P<0.0001, not significant (ns) as determined by unpaired two-tailed t-test unless otherwise stated.

FIG. 3: Identification of inhibitors of epithelial-mesenchymal transition from high-throughput screening of bioactive lipid library with the screening platform. A) Dual-glo Luciferase assay of HMLE-ZEB1 screened with bioactive lipid library. Data are represented as ratio of mean Firefly luciferase (FF) and Renilla luciferase (Ren) readings, n=3. Hits are circled in red. B) Average Z-score of Dual-glo Luciferase assay in HMLE-ZEB1 cells screened with bioactive lipid library; n=4. Compounds shown in red indicates top hits. C) Protein expression of EMT markers E-cadherin, Fibronectin Vimentin and Snail in DMSO and 4-OHT induced cells treated with and without Cay10603 (10 nM) in HMLE-TWIST. GAPDH was used as a loading control. D) (First)Morphological evidence and (second) Fluorescence activated sorting analysis (FACS) of CD44 and CD24 staining in DMSO, 4-OHT, CAY10603 and 4-OHT+CAY10603 treated cells. Numbers within graph represent percentages of gated cells indicating CD44highCD24low (above) and CD44lowCD24high cells (below). E) Representative bright-field images and quantification of DMSO,4-OHT, CAY,4-OHT+CAY treated cells as shown by of scratch wound migration assay before and after 6 h of scratch in presence or absence of 4-OHT-induced EMT. Images were taken at 4× magnification. Scale bar represents 100 μm. F) (First) Representative images of whole-mount fluorescence lung image and (Second) histology of lung sections. Scale bar represents 100 μm. (Third)Tumors were stained for HDAC6 and counterstained with hematoxylin. Scale bar represents 100 μm. (Fourth) Primary tumors were also stained for E-cadherin, Fibronectin and DAPI. Scale bar represents 50 μm. n=4 mice for each group. G) Total number of metastasis as counted from whole mount of lung with −Dox, +Dox and ACY-1215+Dox treatment. n=3-4 for each group. **P<0.01 as determined by as determined by one-way ANOVA with Dunnet's multiple comparison test. All data are presented as mean+/−SEM. *P<0.05, **P<0.01, ***P<0.001 and ****P<0.0001, not significant (ns) as determined by unpaired two-tailed t-test unless otherwise stated.

FIG. 4. Disruption of microtubule depolymerization through HDAC6 inhibition and GSK3β inhibition restricts EMT. A) Top, Phase-contrast images of morphological changes in DMSO and 4-OHT induced cells treated with Ken, ACY-1215 and combination of both in HMLE-TWIST. Arrows represent cells growing as epithelial clusters. Scale represents 100 μm. Second, Immunofluorescence staining of acetylated α-tubulin in DMSO, Ken+ACY-1215, 4-OHT, ACY-1215+4-OHT, Ken+4-OHT and Ken+ACY-1215+4-OHT treated cells. Scale represents 50 μm. (Third) Fluorescence activated sorting analysis (FACS) of CD44 and CD24 staining in DMSO, Ken+ACY-1215, 4-OHT, ACY-1215+4-OHT, Ken+4-OHT and Ken+ACY-1215+4-OHT treated cells. Numbers within graph represent percentages of gated cells indicating CD44highCD24low (above) and CD44lowCD24high cells (below). B) Immunoblotting for E-cadherin, Fibronectin, Snail for DMSO, Ken+ACY-1215, 4-OHT, ACY-1215+4-OHT, Ken+4-OHT and Ken+ACY-1215+4-OHT treated HMLE-TWIST cells. GAPDH was used as a loading control. C) Quantification of protein expression for DMSO, Ken+ACY, 4-OHT, ACY+4-OHT, Ken+4-OHT and Ken+ACY+4-OHT treated HMLE-TWIST cells (n=3). D) Representative bright-field images of scratch assay in DMSO, Ken+ACY-1215, 4-OHT, ACY-1215+4-OHT, Ken+4-OHT and Ken+ACY-1215+4-OHT treated HMLE-TWIST. Images were taken at 4× magnification. Scale bar represents 100 μm. E) Quantification of percentage area closure in DMSO, Ken+ACY-1215, 4-OHT, ACY-1215+4-OHT, Ken+4-OHT and Ken+ACY-1215+4-OHT treated HMLE-TWIST cells. Graph represents mean+/−SEM of n=4. *P<0.05 and **P<0.01 as determined by unpaired t-test (two-tailed) by Welch's correction. F) Total number of metastasis as counted from whole mount of lung in mice implanted with shControl and shGSK3β cells treated with ACY-1215 in presence or absence of Dox treatment (n=3-4). G) Tumor weight of mice implanted with shControl and shGSK3β cells treated with ACY-1215 in presence or absence of Dox treatment (n=4). H) Top, Images of whole-mount of lung. Scale-bar, 100 μm. Second, histology of lung sections. Scale-bar, 100 μm. All data are presented as mean+/−SEM. *P<0.05, **P<0.01, ***P<0.001 and ****P<0.0001, not significant (ns) as determined by unpaired two-tailed t-test unless otherwise stated.

### DETAILED DESCRIPTION

The present specification is directed to a method of inhibiting epithelial-mesenchymal transition (EMT) in a cancer cell, the method comprising contacting the cancer cell with an effective amount of a histone deacetylase 6 (HDAC6) inhibitor or a derivative thereof and/or a glycogen synthase kinase 3β (GSK3β) inhibitor, or a derivative thereof.

The method may comprise contacting the cancer cell with an effective amount of a HDAC6 inhibitor and a GSK3β inhibitor.

The HDAC6 inhibitor may, for example, be ricolinostat (ACY-1215), CAY10603, tubacin, citarinostat (ACY-241), ACY-738, ACY-775, QTX-125, CKD-506, nexturastat A, tubastatin A, or HPOB, or a derivative thereof. Alternatively, the HDAC6 inhibitor may be a nucleic acid inhibitor.

The GSK3β inhibitor may, for example, LY2090314, SAR502250, AZD2858, CHIR-99021, SB 216763, Tideglusib, TWS119, AR-A014418, TDZD-8, GSK 3 Inhibitor IX, Kenpaullone, Cromolyn sodium, CHIR-98014, AZD1080, R547, RGB-286638, 9-ING-41, SB 415286, BRD0705, IM-12, AZD2858, Indirubin-3′-monoxime, 1-Azakenpaullone, CP21R7, Bikinin, BIO-acetoxime, VP3.15 dihydrobromide, GNF4877, GSK-3p inhibitor 1, hSMG-1 inhibitor 11j or a derivative thereof. Alternatively, the GSK3β inhibitor may be a nucleic acid inhibitor.

In one embodiment, there is provided a method of inhibiting epithelial-mesenchymal transition (EMT) in a cancer cell, the method comprising contacting the cancer cell with an effective amount of a histone deacetylase 6 (HDAC6) inhibitor and glycogen synthase kinase 3β (GSK3β) inhibitor.

The method may comprise contacting the cancer cell with an effective amount of ricolinostat (ACY-1215)/CAY10603 and kenpaullone/Indirubin-3′-oxime.

Alternatively, the method may comprise contacting the cancer cell with an effective amount of ricolinostat (ACY-1215) or CAY10603 and a nucleic acid inhibitor of GSK3β.

The method may comprise contacting the cancer cell with an effective amount of ricolinostat (ACY-1215) and a GSK3β inhibitor. The GSK3β inhibitor may, for example, be LY2090314, SAR502250, AZD2858, CHIR-99021, SB 216763, Tideglusib, TWS119, AR-A014418, TDZD-8, GSK 3 Inhibitor IX, Kenpaullone, Cromolyn sodium, CHIR-98014, AZD1080, R547, RGB-286638, 9-ING-41, SB 415286, BRD0705, IM-12, AZD2858, Indirubin-3′-monoxime, 1—Azakenpaullone, CP21R7, Bikinin, BIO-acetoxime, VP3.15 dihydrobromide, GNF4877, GSK-3β inhibitor 1, hSMG-1 inhibitor 11j or a derivative thereof.

In one embodiment, there is provided a method of inhibiting epithelial-mesenchymal transition (EMT) in a cancer cell, the method comprising contacting the cancer cell with an effective amount of ricolinostat (ACY-1215), CAY10603, kenpaullone or Indirubin-3′-oxime, or a derivative thereof.

Without being bound by theory, the inventors present a technology platform for: (i) the identification of gene targets involved in the activation an epithelial-mesenchymal transition (EMT) program and cancer metastasis; (ii) the identification of gene targets, whose inhibition, can prevent EMT and metastasis; and (iii) the identification of chemical compounds, including pathway inhibitors which can prevent EMT and metastasis. Provided herein are therefore also methods for inhibiting EMT in a cancer cell and inhibiting or preventing metastasis of a cancer cell.

As used herein, the term “epithelial-mesenchymal transition” (EMT) refers to the conversion from an epithelial to a mesenchymal phenotype, which is usually a normal process of embryonic development. EMT is also the process whereby injured epithelial cells that function as ion and fluid transporters become matrix remodeling mesenchymal cells. In carcinomas, this transformation typically results in altered cell morphology, the expression of mesenchymal proteins and increased invasiveness. The criteria for defining EMT in vitro involve the loss of epithelial cell polarity, the separation into individual cells and subsequent dispersion after the acquisition of cell motility. Classes of molecules that change in expression, distribution, and/or function during EMT, and that are causally involved, include growth factors (e.g., transforming growth factor (TGF)-β, wnts), transcription factors (e.g., Snail, SMAD, LEF, and nuclear β-catenin), molecules of the cell-to-cell adhesion axis (cadherins, catenins), cytoskeletal modulators (Rho family), and extracellular proteases (matrix metalloproteinases, plasminogen activators).

As used herein, the term “inhibitor” means an agent that decreases or inhibits the function or biological activity of a polypeptide, or the expression of a gene

The term “agent” or “modulatory agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

In some embodiments, the HDAC6 inhibitor or GSK3β inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a nucleic acid molecule, such as an mRNA molecule. In some embodiments, the antisense RNA, siRNA, or shRNA hybridizes to a sequence within a HDAC6 or GSK3β genomic nucleic acid molecule or mRNA molecule and decreases expression of the HDAC6 or GSK3β polypeptide in a cell in the subject. In some embodiments, the HDAC6 or GSK3β inhibitor comprises an antisense RNA that hybridizes to a HDAC6 or GSK3β genomic nucleic acid molecule or mRNA molecule and decreases expression of the HDAC6 or GSK3β polypeptide in a cell in the subject. In some embodiments, the HDAC6 or GSK3β inhibitor comprises an siRNA that hybridizes to a HDAC6 or GSK3β genomic nucleic acid molecule or mRNA molecule and decreases expression of the HDAC6 or GSK3β polypeptide in a cell in the subject. In some embodiments, the HDAC6 or GSK3β inhibitor comprises an shRNA that hybridizes to a HDAC6 or GSK3β genomic nucleic acid molecule or mRNA molecule and decreases expression of the HDAC6 or GSK3β polypeptide in a cell in the subject.

In one embodiment, the inhibitor of HDAC6 or GSK3β is an anti-sense RNA. In one embodiment, the inhibitor of HDAC6 or GSK3β is an siRNA. In one embodiment, the inhibitor of HDAC6 or GSK3β is an shRNA.

The inhibitory nucleic acid molecules disclosed herein can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules disclosed herein can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

As used herein a “small molecule” refers to a compound that has a molecular weight of less than 3 kilodalton (kDa), and typically less than 1.5 kilodalton, and more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kilodalton, less than 1.5 kilodalton, or even less than about 1 kDa.

By “modulating” is meant increasing or decreasing, either directly or indirectly, the level or functional activity of a target molecule. For example, an agent may indirectly modulate the level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

As used herein, the term “function” refers to a biological, enzymatic, or therapeutic function.

The term “derivative” refers to a compound having a structure derived (e.g., by chemical transformation) from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives of small molecules include salts, esters, amides, salts of esters or amides, and N-oxides of a parent compound. With reference to polypeptides, the term “derivative” refers to a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules. The preparation of derivatives can be carried out by methods known in the art.

The terms “polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

The term “pro-drug” is used in its broadest sense and encompasses those derivatives that are converted in vivo to the compounds of the invention. Such derivatives would readily occur to those skilled in the art, and include, for example, compounds where a free hydroxy group is converted into an ester derivative.

In one embodiment, the method comprises contacting the cancer cell with a cancer therapy agent. The cancer therapy agent may be a radiosensitizing agent, a chemotherapeutic agent, a hormone ablation therapy agent, or an immunotherapeutic agent. The cancer therapy agent can be any standard of care chemotherapeutic agent. Chemotherapeutic agents may include, for example, alkylating agents (e.g. cisplatin, cyclophosphamide, carboplatin), antimetabolites (pemetrexed, gemcitabine, flurouracil), anti-microtubule agents (paclitaxel, vinorelbin), topoisomerase inhibitors (irinotecan, etoposide, doxorubicin), and cytotoxic antibiotics (actinomycin).

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

Similarly, a “pharmacologically acceptable” salt, ester, amide, prodrug or derivative of a compound as provided herein is a salt, ester, amide, prodrug or derivative that this not biologically or otherwise undesirable.

The term “expression” refers the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only.

By “expression vector” is meant any genetic element capable of directing the transcription of a polynucleotide contained within the vector and suitably the synthesis of a peptide or polypeptide encoded by the polynucleotide. Such expression vectors are known to practitioners in the art.

The term “gene” as used herein refers to any and all discrete coding regions of the cell's genome, as well as associated non-coding and regulatory regions. The term is intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

The specification is also directed to a method of inhibiting or preventing metastasis of a cancer cell, the method comprising contacting the cancer cell with an effective amount of a histone deacetylase 6 (HDAC6) inhibitor, or a derivative thereof, and/or a glycogen synthase kinase 3β (GSK3β) inhibitor, or a derivative thereof.

In one embodiment, there is provided a method of inhibiting or preventing metastasis of a cancer cell, the method comprising contacting the cancer cell with an effective amount of a histone deacetylase 6 (HDAC6) inhibitor and a glycogen synthase kinase 3β (GSK3β) inhibitor.

In one embodiment, there is provided a method of inhibiting or preventing metastasis of a cancer cell, the method comprising contacting the cancer cell with an effective amount of ricolinostat, CAY10603, kenpaullone or Indirubin-3′-oxime, or a derivative thereof.

The present specification also provides a method of inhibiting the proliferation of a cancer cell, the method comprising contacting an effective amount of a histone deacetylase 6 (HDAC6) inhibitor, or a derivative thereof, and/or a glycogen synthase kinase 33 (GSK3β) inhibitor, or a derivative thereof with the cancer cell.

In one embodiment, there is provided a method of inhibiting the proliferation of a cancer cell, the method comprising contacting an effective amount of a histone deacetylase 6 (HDAC6) inhibitor and a glycogen synthase kinase 3β (GSK3β) inhibitor with the cancer cell

In one embodiment, there is provided a method of inhibiting the proliferation of a cancer cell, the method comprising contacting an effective amount of a histone deacetylase 6 (HDAC6) inhibitor or a glycogen synthase kinase 3β (GSK3β) inhibitor, or a derivative thereof with the cancer cell.

The specification also teaches a method of treating cancer in a subject, the method comprising administering an effective amount of a histone deacetylase 6 (HDAC6) inhibitor, or a derivative thereof, and/or a glycogen synthase kinase 3β (GSK3β) inhibitor, or a derivative thereof, to the subject.

In one embodiment, there is provided a method of treating cancer in a subject, the method comprising administering an effective amount of a histone deacetylase 6 (HDAC6) inhibitor and a glycogen synthase kinase 3β (GSK3β) inhibitor to the subject.

In one embodiment, there is provided a method of treating cancer in a subject, the method comprising administering an effective amount of ricolinostat, CAY10603, kenpaullone or Indirubin-3′-oxime, or a derivative thereof to the subject.

The method may comprise administering an effective amount of ricolinostat/CAY10603 and kenpaullone/Indirubin-3′-oxime to the subject.

The present specification also teaches a method of preventing metastasis of a cancer in a subject, the method comprising administering an effective amount of a histone deacetylase 6 (HDAC6) inhibitor, or a derivative thereof, and/or a glycogen synthase kinase 3β (GSK3β) inhibitor, or a derivative thereof, to the subject.

In one embodiment, there is provided a method of preventing metastasis of a cancer in a subject, the method comprising administering an effective amount of ricolinostat, CAY10603, kenpaullone or Indirubin-3′-oxime, or a derivative thereof to the subject.

The method may comprise administering an effective amount of ricolinostat/CAY10603 and kenpaullone/Indirubin-3′-oxime to the subject.

As used herein, the terms “treatment,” “treating,” and the like, refer to obtaining a desired pharmacologic and/or physiologic effect. The effect may be therapeutic in terms of a partial or complete cure for a disease or condition (e.g., a cancer including a metastatic cancer) and/or adverse effect attributable to the disease or condition. These terms also cover any treatment of a condition or disease in a mammal, particularly in a human, and include: (a) inhibiting the disease or condition, i.e., arresting its development; or (b) relieving the disease or condition, i.e., causing regression of the disease or condition.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer.

In one embodiment, the cancer is a cancer whose metastasis rely on EMT. The cancer may, for example, be breast cancer, lung cancer or liver cancer.

The term “subject” as used throughout the specification is to be understood to mean a human or may be a domestic or companion animal. While it is particularly contemplated that the methods of the invention are for treatment of humans, they are also applicable to veterinary treatments, including treatment of companion animals such as dogs and cats, and domestic animals such as horses, cattle and sheep, or zoo animals such as primates, felids, canids, bovids, and ungulates. The “subject” may include a person, a patient or individual, and may be of any age or gender.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

Provided herein is a pharmaceutical combination comprising a histone deacetylase 6 (HDAC6) inhibitor, or a derivative thereof, and/or a glycogen synthase kinase 3β (GSK3β) inhibitor, or a derivative thereof.

The terms “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together or separately in different compositions. In general, it is expected that additional therapeutic agents utilized in combination be utilized at levels that do not exceed the levels at which they are utilized individually. In some embodiments, the levels utilized in combination will be lower than those utilized individually.

Provided herein is a pharmaceutical combination as defined herein for use as a medicament.

In one embodiment, there is provided a pharmaceutical combination as defined herein for inhibiting epithelial-mesenchymal transition (EMT) in a cancer cell, for inhibiting or preventing metastasis of a cancer cell, for treating cancer in a subject or for preventing metastasis of a cancer in a subject.

In one embodiment, there is provided the use of a pharmaceutical combination as defined herein in the manufacture of a medicament for inhibiting epithelial-mesenchymal transition (EMT) in a cancer cell, for inhibiting or preventing metastasis of a cancer cell, for treating cancer in a subject or for preventing metastasis of a cancer in a subject.

In one embodiment, there is provided a pharmaceutical composition comprising a histone deacetylase 6 (HDAC6) inhibitor, or a derivative thereof, and/or a glycogen synthase kinase 3β (GSK3β) inhibitor, or a derivative thereof.

Disclosed herein is a method of screening for an inhibitor of epithelial-mesenchymal transition (EMT), the method comprising a) contacting a reporter cell line with a library of candidate inhibitors, so as to identify an inhibitor of EMT in the reporter cell line, wherein the reporter cell line comprises a ZEB1 inducible construct and an epithelial gene reporter construct, wherein the reporter cell line is induced to express ZEB1 to promote EMT, and wherein the epithelial gene reporter construct is capable of reporting the inhibition of EMT in the presence of an inhibitor of EMT.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000. By “control element” or “control sequence” is meant nucleic acid sequences (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence.

The term “ZEB1 inducible construct” refers to an inducible construct that can be turned on to express ZEB1 polypeptide. Inducible constructs or systems are well-known in the art. For example, the inducible construct may be a Tet-inducible construct that can be turned on to express ZEB1 polypeptide in the presence of tetracycline.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

Disclosed herein is a reporter cell line comprising a ZEB1 inducible construct. A polynucleotide encoding ZEB1 polypeptide may be inserted into an appropriate expression vector, i.e., a vector which contains the necessary elements for the transcription and translation of the inserted coding sequence, or in the case of an RNA viral vector, the necessary elements for replication and translation. The expression vector may comprise an inducible promoter. The expression vector may then be transfected into a suitable target cell which will express the polypeptide. Transfection techniques known in the art include, but are not limited to, calcium phosphate precipitation (Wigler et al., 1978. Cell 14:725) and electroporation (Neumann et al., 1982. EMBO J. 1:841).

In one embodiment, the nucleic acid or construct is integrated into the genome (e.g. chromosome) of the host cell. Integration can be promoted by inclusion of sequences which promote recombination with the genome, in accordance with standard techniques. In one embodiment, the method as defined herein comprises inducing the reporter cell line to express ZEB1 for a sufficient time to promote EMT in the reporter cell line. The reporter cell line may then be assayed for markers that is indicative of epithelial or mesenchymal cell state. The reporter cell line may further comprise an epithelial gene reporter construct which provides an indication of the epithelial cell state. The reporter cell line may also comprise a mesenchymal gene reporter construct which provides an indication of the mesenchymal cell state.

Disclosed herein is a reporter cell line comprising a ZEB1 inducible construct and an epithelial gene reporter construct.

In one embodiment, the epithelial gene reporter construct is a reporter of cadherin 1 (CDH1) transcription. The epithelial gene reporter construct may comprise a promoter region from the cadherin 1 (CDH1) gene that drives the expression of a luciferase and/or GFP. The luciferase may, for example, be Firefly luciferase.

The ZEB1 inducible construct may be a Tet-inducible ZEB1 construct.

The reporter cell line may further comprise a cell viability reporter construct. This may, for example, be a constitutively expressed Renilla luciferase reporter construct.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

### Results

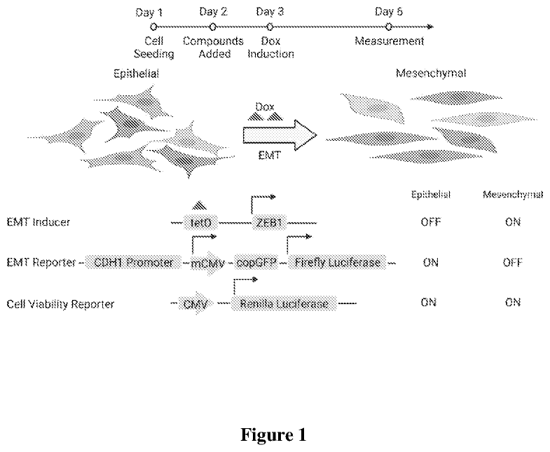

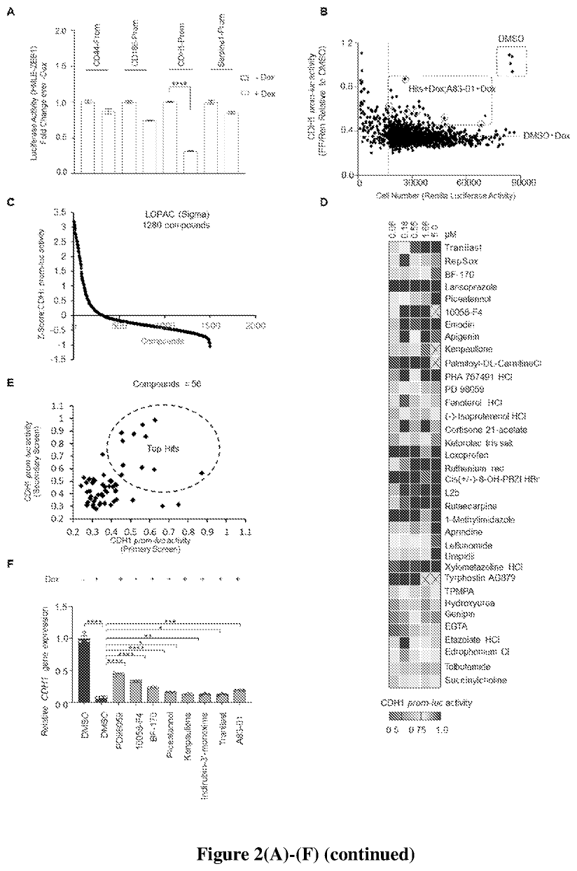

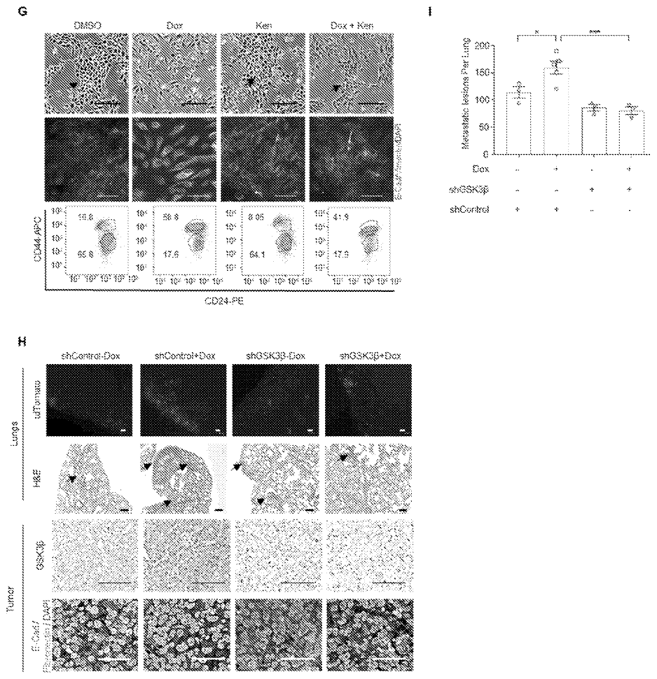

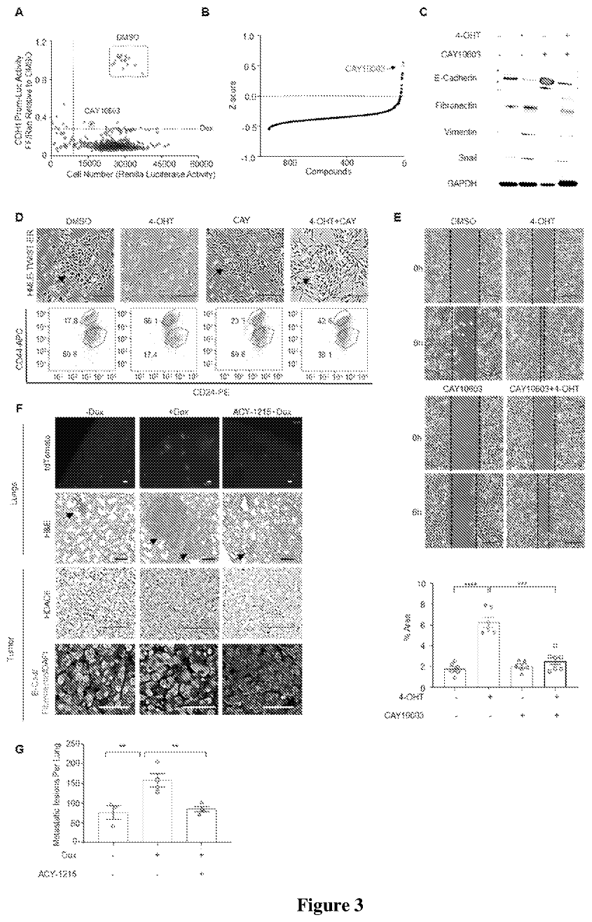

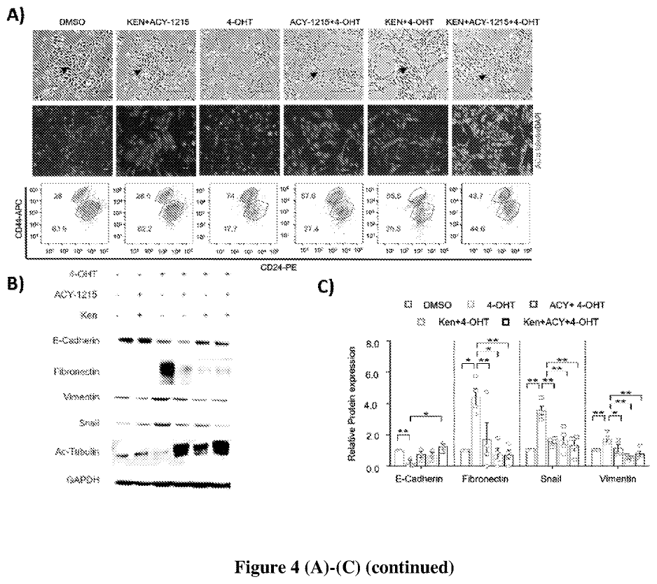

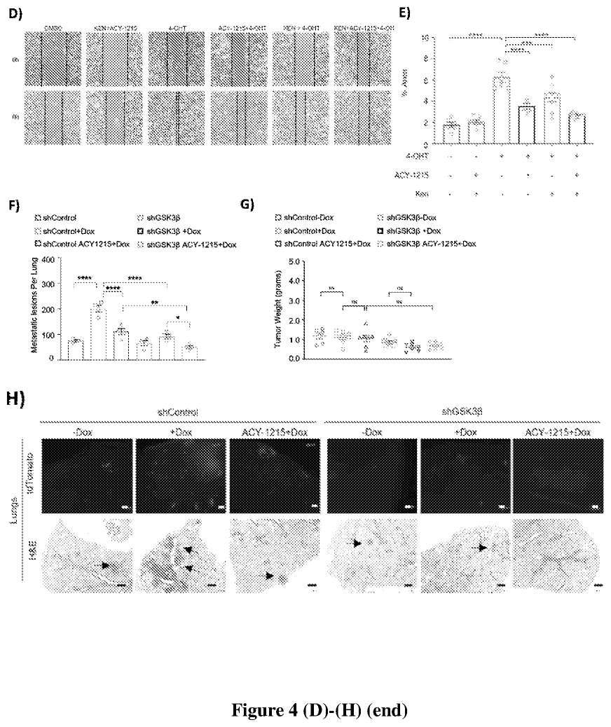

The contributions of EMT as a major cell-biological process involved in the acquisition of cancer stem cell-like properties and promotion of the invasion-metastasis cascade have been well-documented. While studies have demonstrated that the blockade of EMT may be useful for inhibiting metastasis, the therapeutic implications and relevance were less forthcoming¹⁻³. For instance, blockade of TGFβ signaling prevents EMT in vitro but TGFβ inhibitors have pleiotropic effects in both facilitating and arresting cancer progression⁴. To identify compounds and, potentially less well-studied, pathways involved in the regulation of EMT, a cell-based reporter assay has been designed using genetically engineered breast epithelial cells that were amendable to the conditional induction of an EMT program via ZEB1 overexpression. ZEB1 is an EMT transcription factor capable of potently driving cell state transitions when overexpressed^{1,5}. Several EMT associated gene promoters that include CD44, CD166, CDH1 and SERPINE1 coupled to firefly luciferase reporters tagged to promoter regions were tested as readouts for cell state changes in ZEB1-overexpressed HMLE cells. The responsiveness of these gene promoters were evaluated by inducing cells to undergo EMT. CDH1 promoter showed the most significant change in luciferase activity (FIG. 2A). Since CDH1 encodes the epithelial protein E-cadherin, expressed in the epithelial cell state, it is downregulated upon EMT, as well as being lowly or not expressed in mesenchymal-like cells^{6,7}. The same cells were also transduced with a constitutively-expressed Renilla luciferase reporter under the control of a constitutively active CMV promoter; this was used as a readout for cell numbers (FIG. 1). The purpose was to identify compounds and pathway inhibitors that strictly perturbed cell states but did not affect cell viability. Upon induction of an EMT program via ZEB1 overexpression, HMLE-ZEB1 cells underwent an EMT and downregulated CDH1prom-luc reporter by at least 70% (FIG. 2B).

Of the over 2000 compounds screened across two chemical libraries (LOPAC and bioactive lipid library), 78 compounds (hit rate: 3.6%) were able to prevent a reduction of CDH1prom-luc activity (Table 1). 56 compounds from primary screen of LOPAC library which were top hits were also validated by secondary screen whereby 42 compounds were confirmed to be able to prevent the reduction of CDH1prom-luc activity suggesting a robust hit rate of 75% between primary and secondary screen (FIGS. 2B,2C and E). The specificity of the assay was also validated by concentration-dependent effects on CDH1prom-luc activity (FIG. 2D). Upon Dox induction of HMLE-ZEB1 cells gene expression of CDH1 gene was markedly elevated in ZEB1-induced cells treated with EMT inhibitors identified from secondary screen (FIG. 2F). Amongst the candidates, a GSK3β inhibitor, Kenpaullone, appeared as a top anti-EMT candidate. To validate results of the screen, it was confirmed that Kenpaullone was able to inhibit ZEB1-induced EMT, as seen by the retention of an epithelial morphology (FIG. 2G). To assess the targets identified from screening platform, direct functional involvement of GSK3β in metastasis was evaluated. A mouse xenograft model was utilized, whereby human breast cancer cells (MCF7), residing in an epithelial cell state, can be induced to undergo EMT-dependent metastasis in a conditional manner with the doxycycline-inducible expression of both the EMT transcription factors, SLUG and SOX9 (MCF7-SLUG+SOX9)⁸. MCF7-SLUG+SOX9 cells were transduced with GSK3,6 shRNA. Following transplantation in the first group of mice, in the absence of doxycycline treatment, uninduced MCF7-SLUG+SOX9 shControl cells were highly tumorigenic, but otherwise poorly metastatic as expected (FIGS. 2H and 2I). In the second group, MCF7-SLUG+SOX9 shControl mice that were dosed with doxycycline for two weeks to turn on the expression of SLUG+SOX9 had an increase of metastatic lesions in the lungs by ten weeks, despite smaller tumor burdens. In the third and fourth groups, mice implanted with MCF7-SLUG+SOX9 cell bearing GSK3,6 knockdown (shGSK3β) were dosed with doxycycline or water as control. Even in the presence of doxycycline, these mice had comparable number of baseline metastatic lesions as control non-doxycycline treated shControl group, thus confirming that inhibition of GSK3β kinase activity blocked metastasis. This was accompanied by decreased Fibronectin expression, thereby arresting EMT-dependent metastasis.

Amongst the candidates was a HDAC6-specific inhibitor—CAY10603 (FIGS. 3A and B, Table 1). To validate results of the screen, it was confirmed that CAY10603 was able to inhibit EMT driven by another EMT transcription factor, TWIST, as seen by retention of E-cadherin expression, while inhibiting the gain of mesenchymal markers (FIG. 3C). CAY10603-treated cells retained epithelial morphology along with the block in gain of CD44 expression to some extent, typically associated with a mesenchymal cell state following EMT (FIG. 3D). CAY10603-treated cells also blocked the acquisition of migration ability, typically associated with a mesenchymal cell state, as measured by wound-healing assay (FIG. 3E).

To test the anti-metastatic effect of HDAC6 as a target obtained from the screening platform, a HDAC6 pharmacological inhibitor, ACY-1215 (also known as ricolinostat) which has been used in several clinical trials, was used. When xenografted into the fat-pads of immunodeficient NSG mice, in the absence of doxycycline, MCF7-SLUG+SOX9 cells were highly tumorigenic but otherwise poorly metastatic, owing to the absence of an activated EMT program⁸. Upon doxycycline induction, metastatic lesions could be observed in the lungs (FIGS. 3F and 3G). To evaluate the direct function of HDAC6, ACY-1215, which is not limited by in vivo bioavailability was used, and its impact on EMT-driven breast cancer metastasis was explored⁹. Here, MCF7-SLUG+SOX9 cells were transplanted orthotopically into the fat-pads of NSG mice, and they were randomized into three groups (−Dox, +Dox and ACY-1215+Dox). Mice were administered vehicle control (5% DMSO in corn oil; −Dox) or ACY-1215 (50 mg/kg) via intraperitoneal (i.p.) administration, which commenced 5 days before and continued throughout the duration of doxycycline administration. Doxycycline was administered by drinking water containing 1 mg/ml doxycycline and 10 mg/ml sucrose for 10 days.

Mice were sacrificed 10 weeks after doxycycline treatment and analyzed for lung metastases and tumor burden. Compared to doxycycline-fed mice whose tumor cells underwent EMT, ACY-1215 drug-treated mice showed significantly reduced lung metastatic lesions (FIG. 3F-G). Tumors harvested from Dox-fed mice displayed higher expressions of HDAC6 and Fibronectin and low expression of E-Cadherin, which were consistent with the activation of an EMT within primary tumors in the mammary glands. This observation was strikingly absent in control and ACY-1215-treated groups. Thus, HDAC6 appeared necessary for the activation of EMT to spawn metastases, and its pharmacological inhibition might be crucial for controlling metastasis in breast cancer.

To test any synergistic effect, the effects of disrupting of both GSK3β and HDAC6-mediated pathways in cell-state transition and metastasis were examined. Indeed, pharmacological inhibition of both pathways with Kenpaullone and ACY-1215 had a synergistic effect on microtubule depolymerization as measured by acetylated tubulin expression in Twist induced HMLE cells (FIGS. 4A and 4B). This was also associated with higher E-cadherin and CD24 expression, along with inhibition of activation of mesenchymal markers such as CD44 (FIG. 4C). In addition, wound migration assay showed that the combination could inhibit EMT-dependent migration most significantly (FIG. 4D-E).

When xenografted into the fat-pads of immunodeficient NSG mice and upon doxycycline induction, metastatic lesions could be observed in the lungs; this was reduced with ACY-1215 treatment in shControl group. Compared to Dox-fed mice in shControl group, treatment with ACY-1215 in the shGSK3β group reduced metastatic lesions most dramatically, despite non-significant changes in tumor burden (FIG. 4F-H). Taken together, these data indicated the therapeutic benefit of targeting microtubule regulation by both pathways in controlling metastasis in breast-cancer.

TABLE 1
List of top EMT-inhibiting compounds from primary
screen of LOPAC and bioactive lipid library

| | | | DMSO normalized |
|---|---|---|---|
| Library | Compound | RL-average | FF/RL(DMSO = 1) |
| Lopac | Kenpaullone | 25920 | 0.871498671 |
| Lopac | Emodin | 33922.33333 | 0.626778437 |
| Lopac | Piceatannol | 17037.75 | 0.62527606 |
| Lopac | Apigenin | 26889.33333 | 0.578660026 |
| Lopac | 10058-F4 | 16782 | 0.563081682 |
| Lopac | Rutaecarpine | 32323 | 0.559736055 |
| Lopac | BF-170 | 47781 | 0.517350967 |
| | hydrochloride | | |
| Lopac | PD 98,059 | 48143.75 | 0.459165912 |
| Lopac | Tranilast | 68070.25 | 0.452675858 |
| Lipid | CAY 10603 | 16158 | 0.33 |
| Lipid | 2-O-methyl | 13317 | 0.35 |
| | PAF C-18 | | |
| Lipid | Pifithrin-α | 26922.5 | 0.348 |

### REFERENCES

1 Krebs, A. M. et al. The EMT-activator Zeb1 is a key factor for cell plasticity and promotes metastasis in pancreatic cancer. Nat Cell Biol 19, 518-529, doi:10.1038/ncb3513 (2017).
2 Tsai, J. H. & Yang, J. Epithelial-mesenchymal plasticity in carcinoma metastasis. Genes Dev 27, 2192-2206, doi:10.1101/gad.225334.113 (2013).
3 Tsai, J. H., Donaher, J. L., Murphy, D. A., Chau, S. & Yang, J. Spatiotemporal regulation of epithelial-mesenchymal transition is essential for squamous cell carcinoma metastasis. Cancer Cell 22, 725-736, doi:10.1016/j.ccr.2012.09.022 (2012).
4 Siegel, P. M., Shu, W., Cardiff, R. D., Muller, W. J. & Massague, J. Transforming growth factor beta signaling impairs Neu-induced mammary tumorigenesis while promoting pulmonary metastasis. Proc Natl Acad Sci USA 100, 8430-8435, doi:10.1073/pnas.0932636100 (2003).
Larsen, J. E. et al. ZEB1 drives epithelial-to-mesenchymal transition in lung cancer. J Clin Invest 126, 3219-3235, doi:10.1172/JCI76725 (2016).
6 Eger, A. et al. DeltaEF1 is a transcriptional repressor of E-cadherin and regulates epithelial plasticity in breast cancer cells. Oncogene 24, 2375-2385, doi:10.1038/sj.onc.1208429 (2005).
7 Batlle, E. et al. The transcription factor snail is a repressor of E-cadherin gene expression in epithelial tumour cells. Nat Cell Biol 2, 84-89, doi:10.1038/35000034 (2000).
8 Guo, W. et al. Slug and Sox9 cooperatively determine the mammary stem cell state. Cell 148, 1015-1028, doi:10.1016/j.cell.2012.02.008 (2012).
9 Hideshima, T. et al. Discovery of selective small-molecule HDAC6 inhibitor for overcoming proteasome inhibitor resistance in multiple myeloma. Proc Natl Acad Sci USA 113, 13162-13167, doi:10.1073/pnas.1608067113 (2016).

1-16. (canceled)

17. A method of inhibiting epithelial-mesenchymal transition (EMT) in a cancer cell, the method comprising contacting the cancer cell with an effective amount of a histone deacetylase 6 (HDAC6) inhibitor and a glycogen synthase kinase 3β (GSK3β) inhibitor.

18. The method of claim 17, wherein the HDAC6 inhibitor is ricolinostat (ACY-1215), CAY10603, tubacin, citarinostat (ACY-241), ACY-738, ACY-775, QTX-125, CKD-506, nexturastat A, tubastatin A or HPOB, or wherein the HDAC6 inhibitor is a nucleic acid inhibitor.

19. The method of claim 17, wherein the HDAC6 inhibitor is ricolinostat or CAY10603.

20. The method of claim 17, wherein the GSK3β is a nucleic acid inhibitor, or wherein the GSK3β inhibitor is LY2090314, SAR502250, AZD2858, CHIR-99021, SB 216763, Tideglusib, TWS119, AR-A014418, TDZD-8, GSK 3 Inhibitor IX, Kenpaullone, Cromolyn sodium, CHIR-98014, AZD1080, R547, RGB-286638, 9-ING-41, SB 415286, BRD0705, IM-12, AZD2858, Indirubin-3′-monoxime, 1-Azakenpaullone, CP21R7, Bikinin, BIO-acetoxime, VP3.15 dihydrobromide, GNF4877, GSK-3β inhibitor 1 or hSMG-1 inhibitor 11j.

21. The method of claim 17, wherein the HDAC6 inhibitor is ricolinostat and the GSK3β inhibitor is a nucleic acid inhibitor.

22. The method of claim 17, wherein the method comprises contacting the cancer cell with a cancer therapy agent.

23. The method of claim 22, wherein the cancer therapy agent is selected from an alkylating agent, an antimetabolite, an anti-microtubule agent, a topoisomerase inhibitor or a cytotoxic antibiotic.

24. The method of claim 17, wherein the cancer is breast cancer, lung cancer or liver cancer.

25. The method of claim 17, wherein the cancer is a breast cancer.

26. The method of claim 17, wherein the method inhibits or prevents metastasis of the cancer cell.

27. A method of treating cancer in a subject, the method comprising administering an effective amount of a histone deacetylase 6 (HDAC6) inhibitor and a glycogen synthase kinase 3β (GSK3β) inhibitor to the subject.

28. The method of claim 27, wherein the method inhibits or prevents metastasis of the cancer in the subject.

29. A pharmaceutical combination comprising a histone deacetylase 6 (HDAC6) inhibitor and a glycogen synthase kinase 3β (GSK3β) inhibitor.

30. The pharmaceutical combination of claim 29, wherein the HDAC6 inhibitor is ricolinostat (ACY-1215), CAY10603, tubacin, citarinostat (ACY-241), ACY-738, ACY-775, QTX-125, CKD-506, nexturastat A, tubastatin A or HPOB, or wherein the HDAC6 inhibitor is a nucleic acid inhibitor.

31. The pharmaceutical combination of claim 29, wherein the GSK3β is a nucleic acid inhibitor, or wherein the GSK3β inhibitor is LY2090314, SAR502250, AZD2858, CHIR-99021, SB 216763, Tideglusib, TWS119, AR-A014418, TDZD-8, GSK 3 Inhibitor IX, Kenpaullone, Cromolyn sodium, CHIR-98014, AZD1080, R547, RGB-286638, 9-ING-41, SB 415286, BRD0705, IM-12, AZD2858, Indirubin-3′-monoxime, 1-Azakenpaullone, CP21R7, Bikinin, BIO-acetoxime, VP3.15 dihydrobromide, GNF4877, GSK-3β inhibitor 1 or hSMG-1 inhibitor 11j.

32. The pharmaceutical combination of claim 29, wherein the HDAC6 inhibitor is ricolinostat and the GSK3β inhibitor is a nucleic acid inhibitor.
